(19) United States
(12) Patent Application Publication
(10) Pub. No.: US 2013/0232854 A1
(43) Pub. Date: Sep. 12, 2013

(54) PROCESS FOR SEPARATING FURFURAL FROM A LIQUID AQUEOUS PHASE COMPRISING FURFURAL AND ONE OR MORE ORGANIC ACIDS

(71) Applicant: Johannes Pieter Haan, Amsterdam (NL)

(72) Inventor: Johannes Pieter Haan, Amsterdam (NL)

(21) Appl. No.: 13/698,364

(22) PCT Filed: Jun. 22, 2011
(86) PCT No.: PCT/EP2011/060409

§ 371 Date: Jan. 7, 2013

(30) Foreign Application Priority Data
Jun. 22, 2010 (EP) 10166868.9

(51) Int. Cl. C07D 307/48

(52) U.S. Cl. CPC C07D 307/48
USPC 44/307; 549/490

(57) ABSTRACT

A process for separating furfural from a liquid aqueous phase comprising furfural and one or more organic acids, which process comprises a step a) extracting furfural from the liquid aqueous phase into a liquid aromatic phase comprising one or more aromatic hydrocarbon compounds to obtain a liquid organic phase comprising furfural and one or more aromatic hydrocarbon compounds.

### TECHNICAL FIELD OF THE INVENTION

The present invention relates to a process for separating furfural from a liquid aqueous phase comprising furfural and one or more organic acids.

### BACKGROUND TO THE INVENTION

Furfural, also known as Furan-2-carbaldehyde, is a valuable intermediate in the production of sustainable biofuels. Biofuels are combustible fuels, that can be derived from biological sources. The use of such biofuels results in a reduction of greenhouse gas emissions. Furfural can conveniently be produced from biomass. It is for example produced in the liquefaction of lignocellulosic material.

After the liquefaction of lignocellulosic material it is desirable to separate furfural from the total aqueous product produced. Separation of the furfural by distillation, however, is problematic as furfural can form azeotropes with the water in the total aqueous product.

Alternative approaches to recovering furfural include liquid-liquid extraction processes.

U.S. Pat. No. 4,533,743 describes a process for the production of furfural. It describes that state of the art biomass acid hydrolysis processing techniques can breakdown pentosans, a major constituent of biomass hemicellulose, into pentoses. Hot pentose is subsequently reacted in the presence of a mineral acid catalyst in a plug flow reactor at a temperature in the range from 220° C. to 300° C. to produce furfural. The produced furfural is optionally extracted using an essentially water immiscible furfural solvent which does not form an azeotrope with furfural.

As suitable solvents amongst others higher boiling point aromatics, such as diethylbenzene, dipropylbenzene, dimethylethylbenzene, butylbenzene, tetralin and isophorone; aromatics, such as toluene; halogenated aromatics; and also halogenated alkanes are mentioned. U.S. Pat. No. 6,441,202 describes a method to produce sugars by acidic hydrolysis of biomass and subsequently subject the sugars to dehydration to form a hydrolysate comprising heterocyclic compounds such as furfural and hydroxymethylfurfural and acid. Subsequently the heterocyclic compounds are extracted from the hydrolysate by a hydrocarbon. The acid may include an organic or inorganic acid, such as for example sulfuric acid and the hydrocarbon may for example be toluene. FR2411184 also describes a process for the preparation of furfural. It describes submitting of a sugar solution to acid dehydration to convert xylose into furfural. The furfural is extracted with a solvent. As suitable solvents amongst others toluene, xylene, methylnaphthalene and benzaldehyde are mentioned.

J. Croker et al. describe a process for liquid extraction of furfural from an aqueous solution (see their article “liquid extraction of furfural from aqueous solution” by John R. Croker and Ron G. Bowrey, Ind. Eng. Chem. Fundam. 1984, vol. 23, pages 480-484). They describe extraction for water-furfural methyl isobutyl ketone; water-furfural-isobutyl acetate and water-furfural-toluene systems.

In the above prior art methods for extracting furfural, however, the starting solution is a solution comprising only a limited amount of components, such as for example furfural, water and optionally sugars and/or an acid such as sulfuric acid.

### SUMMARY OF THE INVENTION

It has now been advantageously found that furfural can also be selectively extracted by liquid-liquid extraction from a complex starting solution, comprising not only furfural, water and optionally sugars, but also a complex combination of one or more organic acids.

The present invention therefore provides a process for separating furfural from a liquid aqueous phase comprising furfural and one or more organic acids, which process comprises a step

a) extracting furfural from the liquid aqueous phase into a liquid aromatic phase comprising one or more aromatic hydrocarbon compounds to obtain a liquid organic phase comprising furfural and one or more aromatic hydrocarbon compounds.

It has presently been found that contacting the liquid aqueous phase comprising furfural and one or more organic acids with a liquid aromatic phase comprising one or more aromatic hydrocarbon compounds allows one to selectively extract furfural from the liquid aqueous phase into the liquid aromatic phase, leaving the aqueous phase depleted in furfural, but still rich in organic acid.

The present invention, therefore, makes it possible to directly extract furfural from a liquid aqueous phase, comprising furfural and one or more organic acids, formed by acid catalyzed hydrolysis of biomass.

The selective extraction of furfural from organic acids in an aqueous environment has not previously been suggested.

### BRIEF DESCRIPTION OF THE DRAWINGS

FIG. 1 shows a schematic diagram of a process for extracting furfural according to the invention.

FIG. 2 shows a schematic diagram of a process for extracting furfural according to the invention with recovery of the extraction solvent.

### DETAILED DESCRIPTION OF THE INVENTION

In the process according to the present invention, furfural is extracted from a liquid aqueous phase comprising furfural and one or more organic acids.

By a liquid phase is herein understood a phase that is liquid at extraction temperature and pressure. By a liquid aqueous phase is herein understood a liquid phase comprising at least water.

In one embodiment, the liquid aqueous phase is produced by acid catalyzed hydrolysis of biomass, preferably of lignocellulosic material. Preferably the liquid aqueous phase is obtained by liquefaction of lignocellulosic material. As used herein, lignocellulosic material refers to a material comprising lignin, cellulose and hemicellulose.

Without wishing to be bound to any kind of theory it is believed that liquefaction of lignocellulosic material can comprise cleavage of covalent linkages in the cellulose, hemicellulose and lignin present and/or cleavage of covalent linkages between lignin, hemicelluloses and/or cellulose. As a result acids such as formic acid, acetic acid and/or levulinic acid can be formed together with sugars and optionally lignin degradation products. Cellulose present in the lignocellulosic material can be converted into a sugar containing six carbon atoms which is degraded in the presence of the acid catalyst to produce hydroxymethylfurfural and then further hydrolyzed to give levulinic acid. Hemicellulose present in the lignocellulosic material can be converted into a five carbon sugar which breaks down to give furfural.

The lignocellulosic material may be obtained from a variety of plants and plant materials including agricultural wastes, forestry wastes and sugar processing residues. Examples of suitable lignocellulosic materials include agricultural wastes such as corn stover, soybean stover, corn cobs, rice straw, rice hulls, oat hulls, corn fibre, cereal straws such as wheat, barley, rye and oat straw; grasses; forestry products such as wood and wood-related materials such as sawdust; sugar processing residues such as bagasse and beet pulp; or mixtures thereof.

Liquefaction of the lignocellulosic material can be carried out in any manner known to the skilled person to be suitable for such a purpose. The liquefaction of lignocellulosic material can suitably comprise hydrolyzing the lignocellulosic material in the presence of an acid catalyst. Examples of suitable acid catalysts include mineral acids such as sulphuric acid, para-toluene-sulphonic-acid, nitric acid, hydrochloric acid, phosphoric acid and/or mixtures thereof; and organic acids such as oxalic acid, formic acid, lactic acid, citric acid, trichloracetic acid and/or mixtures thereof. The temperature applied during hydrolysis may vary widely and preferably ranges from 100° C. to 300° C., more preferably from 150° C. to 250° C.

In one embodiment, a lignocellulosic material is hydrolyzed in the presence of an acid catalyst acid to prepare a liquid aqueous phase comprising furfural and one or more organic acids, which liquid aqueous phase is subsequently used directly as the starting liquid aqueous phase of the process according to the invention in the absence of additional recovery or concentration steps. The process of the invention is therefore particularly advantageous for the extraction of furfural from a liquid aqueous phase comprising furfural and one or more organic acids, which liquid aqueous phase is produced by acid catalyzed hydrolysis of a lignocellulosic material.

The liquid aqueous phase comprises preferably more than or equal to 0.1 wt %, more preferably more than or equal to 0.5 wt %, and most preferably more than or equal to 1.0 wt % furfural and preferably less than or equal to 30.0 wt %, more preferably less than or equal to 20 wt % and most preferably less than or equal to 10.0 wt % furfural, based on the total weight of the liquid aqueous phase.

The liquid aqueous phase further comprises preferably more than or equal to 0.1 wt %, more preferably more than or equal to 0.5 wt %, and most preferably more than or equal to 1.0 wt % of one or more organic acids and preferably less than or equal to 30.0 wt %, more preferably less than or equal to 25.0 wt % and most preferably less than or equal to 20.0 wt % of one or more organic acids, based on the total weight of the liquid aqueous phase.

Preferably the one or more organic acids are chosen from the group of levulinic acid, acetic acid, formic acid and mixtures thereof. More preferably the liquid aqueous phase comprises essentially only organic acids chosen from the group of levulinic acid, acetic acid, formic acid and mixtures thereof. In a most preferred embodiment the liquid aqueous phase comprises at least levulinic acid.

By a liquid aromatic phase is herein understood a liquid phase comprising at least one aromatic hydrocarbon compound. In the process according to the invention the liquid aromatic phase can comprise one or more aromatic hydrocarbon compounds.

As used herein, an aromatic hydrocarbon compound is understood to be a compound that comprises a benzene or naphthalene ring, which ring is optionally substituted by one or more alkyl groups. The term “alkyl” includes both straight chain and branched chain alkyl groups. If substituted, the benzene or naphthalene ring is preferably substituted with one to four, more preferably one or two alkyl groups. Preferably the alkyl group contains 1 to 6, more preferably 2 to 4 carbon atoms. Preferably the liquid aromatic phase includes a C₁₋₆ alkyl substituted benzene or a C₁₋₆ alkyl substituted naphthalene or a mixture thereof.

The one or more aromatic hydrocarbon compounds are suitably capable to act as a solvent in which furfural is soluble (at a extraction temperature and pressure) and are preferably substantially water-immiscible. A substantially water-immiscible aromatic hydrocarbon compound refers to an aromatic hydrocarbon compound having a solubility in water of less than 500 mg/kg, at at ambient temperature (20° C.) and pressure (1 bar absolute).

Preferably the liquid aromatic phase comprises one or more aromatic hydrocarbon compounds having a higher boiling point than furfural. The use of one or more aromatic hydrocarbon compounds having a higher boiling point than furfural advantageously facilitates recycling of the aromatic hydrocarbon compound, reducing the amount of energy required and thereby improving the cost effectiveness of the process of the invention.

It has been found that although toluene (methyl benzene) can selectively extract furfural from a liquid aqueous phase comprising furfural and one or more organic acids, the fact that it has a boiling point (111° C.) which is below that of furfural (162° C.) makes it costly to recycle.

Preferred aromatic hydrocarbon compounds include alkyl benzenes having an alkyl side chain with at least 2 carbon atoms and which has a boiling point above that of furfural, such as butyl benzene (boiling point 183° C.), pentylbenzene (boiling point 205° C.) and hexylbenzene (boiling point 226° C.).

Naphthalene has a boiling point (218° C.) which is higher than that of furfural, indicating that it should be well suited to extracting furfural from an aqueous phase comprising furfural. However, the practical applications of naphthalene for use as an extraction solvent (i.e. as liquid aromatic phase) according to the present invention are limited as naphthalene is a solid below 80° C., meaning that liquid/liquid extractions using naphthalene are not feasible below this temperature.

Preferred aromatic hydrocarbon compounds include alkyl substituted naphthalenes, which melt at lower temperatures that naphthalene. In one particular embodiment, the aromatic hydrocarbon compound comprises 1-methylnaphthalene.

Mixtures of aromatic hydrocarbon compounds may also suitably be used in the process of the invention.

In one embodiment, the liquid aromatic phase comprises a mixture of naphthalene and 1-methylnaphthalene. By dissolving naphthalene in 1-methylnaphthalene, which is a liquid at room temperature with a boiling point of 241° C., high extraction selectivity can be achieved whilst avoiding the practical problems associated with using naphthalene alone. In a preferred embodiment the liquid aromatic phase consists essentially of a mixture of naphthalene and 1-methylnaphthalene. Preferably a mixture is used with a weight ratio of naphthalene to 1-methylnaphthalene in the range of 1:5 to 5:1, more preferably 1:4 to 4:1, and most preferably 1:2 to 2:1. The liquid aromatic phase comprises preferably more than or equal to 40 wt %, more preferably more than or equal to 50 wt % and most preferably more than or equal to 60 wt % of one or more aromatic hydrocarbon compounds, based on the total weight of liquid aromatic phase. For practical purposes the liquid aromatic phase may comprise equal to or less than 100 wt % of one or more aromatic hydrocarbon compounds, based on the total liquid aromatic phase.

The liquid aromatic phase further comprises preferably less than 40 wt % of non-aromatic hydrocarbon compounds, more preferably less than 10 wt % of non-aromatic hydrocarbon compounds, based on the total liquid aromatic phase. Most preferably the liquid aromatic phase is essentially free of non-aromatic hydrocarbon compounds. Examples of such non-aromatic hydrocarbon compounds include (branched-)paraffins, (branched-)olefins and naphtenics.

The furfural can be extracted from the liquid aqueous phase comprising furfural and one or more organic acids by contacting the liquid aqueous phase with the liquid aromatic phase comprising one or more aromatic hydrocarbon compounds. The liquid aromatic phase can be added to the liquid aqueous phase or the liquid aqueous phase can be added to the liquid aromatic phase.

Preferably the liquid aromatic phase is added to the liquid aqueous phase. Contacting of the liquid aqueous phase and the liquid aromatic phase can be carried out batchwise or continuously. When carried out continuously the contacting of the liquid aqueous phase and the liquid aromatic phase may be carried out counter-currently, co-currently or cross-currently with respect to each other. Preferably, the liquid aqueous phase and the liquid aromatic phase are contacted with each other counter-currently, more preferably with the liquid stream with the highest density entering at the top of an extraction column and the liquid stream with the lower being fed in at the bottom of the extraction column. Alternatively several mixer/settler units in series can be used.

Upon contact of the liquid aqueous phase and the liquid aromatic phase, two phases can be formed. Furfural present in the liquid aqueous phase is selectively extracted into the liquid aromatic phase, forming a liquid organic phase and leaving the liquid aqueous phase depleted in furfural. Organic acid(s) present in the liquid aqueous phase remain in the liquid aqueous phase, leaving the liquid aqueous phase rich in organic acid(s). By a liquid organic phase is understood a liquid phase comprising one or more organic compounds. The liquid organic phase formed comprises furfural and one or more aromatic hydrocarbon compounds. The formed liquid organic phase comprises preferably in the range from 0.2 wt % to 40.0 wt %, more preferably in the range from 1.0 wt % to 20.0 wt % of furfural based on the total weight of liquid organic phase.

Preferably, the liquid aqueous phase comprising furfural and one or more organic acids and the liquid aromatic phase comprising one or more aromatic hydrocarbon compounds are contacted at a ratio of the liquid aqueous phase to the liquid aromatic phase in the range of from 2:1 to 1:5.

The pressure and temperature can be selected such that the liquid aqueous phase and liquid aromatic phase remain in the liquid state. Preferably the process according to the invention is carried out at a temperature of less than or equal to 200° C., more preferably a temperature of less than or equal to 150° C., most preferably a temperature of less than or equal to 90° C. and a temperature of more than or equal to 15° C., more preferably a temperature of more than or equal to 20° C. The process according to the invention is further preferably conducted at a pressure of less than or equal to 25 bar absolute, more preferably less than or equal to 10 bar, most preferably at ambient pressure (1 bar absolute).

After extraction of the furfural from the liquid aqueous phase into the liquid aromatic phase, the formed furfural-depleted liquid aqueous phase may be separated and removed from the formed liquid organic phase by conventional techniques such as gravitational separation (for example settling).

The formed furfural-depleted liquid aqueous phase is preferably removed from the formed liquid organic phase and preferably recycled and re-used, for example in acid-catalyzed hydrolysis of lignocellulose to provide more furfural in liquid aqueous solution.

The aromatic hydrocarbon compounds are preferably recovered from the formed liquid organic phase and recycled for use in the extraction process.

In a preferred embodiment the process according to the invention subsequently comprises a further step wherein the furfural is retrieved from the liquid organic phase. Separation of the extracted furfural from the liquid organic phase into which it is extracted can be achieved by any technique known to be suitable for this purpose. A preferred technique is distillation. An advantage of using one or more aromatic hydrocarbon compounds having a higher boiling point than furfural is that the extracted furfural can be evaporated instead of the aromatic hydrocarbon compound(s). As the furfural is generally present in a smaller amount than the aromatic hydrocarbon compound(s), this requires less energy. Furthermore, the use of one or more higher boiling point aromatic hydrocarbon compound(s) allows a lower ratio of the liquid aqueous phase to liquid aromatic phase to be used in the process of the invention without incurring a large energy penalty in the recovery step. This means that more aromatic hydrocarbon compound per unit of furfural is available in the extraction tower to remove furfural from the liquid aqueous phase. Additionally, separation of furfural from the liquid organic phase by removal of the furfural (by evaporation from the top of the distillation column) to leave behind the a liquid aromatic phase again affords the advantage that the formation of furfural condensation products is minimized, leading to a higher purity product.

In a preferred embodiment the process according to the invention further comprises a step wherein the retrieved furfural is converted into a furfural derivative, such as for example methyltetrahydrofuran or methylfuran, and wherein this furfural derivative is blended with one or more other fuel components to produce a biofuel.

In another preferred embodiment, the process according to the invention further comprises a step wherein furfural is decarbonylated to furan, which furan is subsequently hydrogenated to tetrahydrofuran and/or hydrated to butanediol. The tetrahydrofuran and butanediol may be used as a chemical.

Hence, the present invention also provides a process for producing tetrahydrofuran and/or butanediol, comprising:
extracting furfural from a liquid aqueous phase, which liquid aqueous phase comprises furfural and optionally one or more organic acids, into a liquid aromatic phase, which liquid aromatic phase comprises one or more aromatic hydrocarbon compounds, to obtain a liquid organic phase comprising furfural and one or more aromatic hydrocarbon compounds;
retrieving furfural from the liquid organic phase to obtain furfural;
decarbonylating the furfural to obtain furan; and
hydrogenating the furan to obtain tetrahydrofuran and/or hydrating the furan to obtain butanediol.

The extraction step and the retrieval step are preferably carried out as described herein above.

The furfural decarbonylation may be carried out in the presence of a decarbonylation catalyst. The decarbonylation catalyst may be any decarbonylation catalyst known by the skilled person in the art to be suitable for decarbonylation.

For example, the decarbonylation catalyst may comprise a metal oxides and one or more catalytic metals. Metal oxide catalysts, for example based on iron, zinc, magnesium, chromium, cobalt, molybdenum or nickel, are preferably used at temperatures of 300° C. to 500° C.

In another example the decarbonylation catalyst may comprise supported noble metals such as Pd/Al₂O₃. The supported noble metal catalysts are preferably used at a temperature of 240° C. to 400° C. under H₂ flow.

In yet another example the decarbonylation is carried out in the liquid phase in the absence of H₂, using an Alumina- or Carbon-supported Pd catalysts and K₂CO₃ as co-catalyst, preferably at a temperature in the range from 100° C. to 250° C.

A subsequent hydrogenation of furan to tetrahydrofuran may be carried out in the presence of a Ni-catalyst.

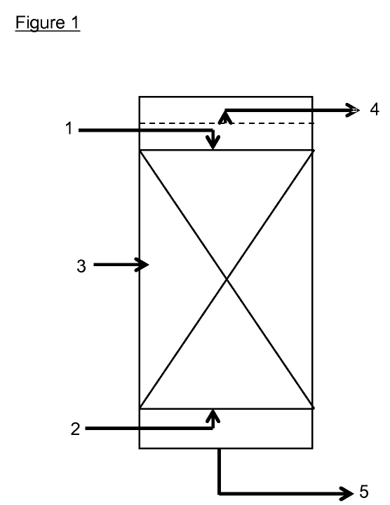

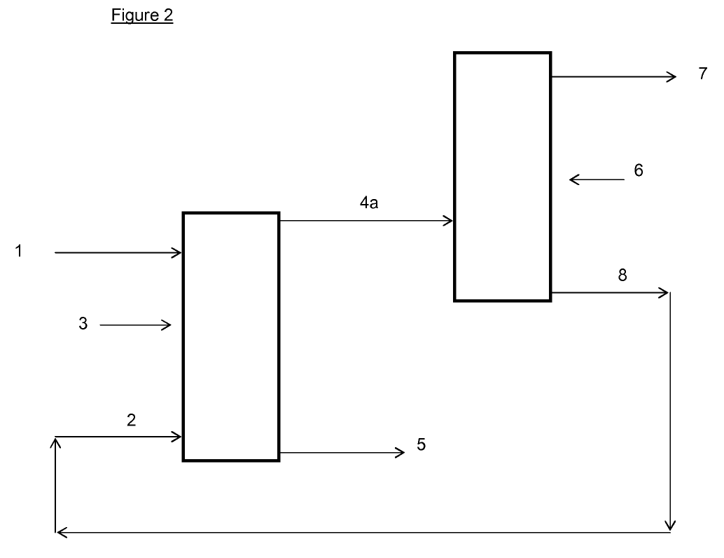

FIGS. 1 and 2 show process schemes for two embodiments of the process according to the invention. In the embodiment shown in FIG. 1, a liquid aqueous phase stream, comprising furfural and at least one of levulinic acid, formic acid and acetic acid, which stream is obtained from an acid-catalyzed hydrolysis of lignocellulosic material, (flow 1), is directed to extraction column (3). An liquid aromatic phase (2) is also introduced into the extraction column (3). In the embodiment shown, the liquid aqueous phase and the liquid aromatic phase are introduced into the extraction column at opposite ends and flow counter-currently with respect to each other, the higher density liquid stream (liquid aqueous phase) entering at the top of the column and the lower density liquid stream (liquid aromatic phase) entering at the bottom of the extraction column (3). In extraction column (3), furfural is extracted from the liquid aqueous phase into the liquid aromatic phase to provide a liquid aqueous phase depleted in furfural and an liquid organic phase comprising furfural and one or more aromatic hydrocarbon compounds. These two phases have different densities and may be separated by gravitational separation as shown in FIG. 1 where the heavier liquid aqueous phase depleted in furfural is withdrawn from the bottom of the extraction column (flow 5) and the lighter liquid organic phase enriched in furfural (flow 4) is removed as the overhead product. In the embodiment shown in FIG. 2, the extraction process is performed using an aromatic hydrocarbon compound having a higher boiling point than furfural. The liquid organic phase enriched in furfural and comprising this aromatic hydrocarbon compound having a higher boiling point than furfural (4a) is then supplied to a distillation column (6) where the lower boiling point (and therefore more volatile) furfural is separated from the higher boiling point aromatic hydrocarbon compound by distillation. Furfural is obtained as a vapour (flow 7) in the overhead of the distillation column and the vapour is condensed to form liquid furfural (not shown). The aromatic hydrocarbon compound is removed from the bottom of the distillation column (flow 8) and recycled back to the extraction column as (part of) the liquid aromatic phase (2).

Throughout the description and claims of this specification, the words “comprise” and “contain” and variations of the words, for example “comprising” and “comprises”, mean “including but not limited to”, and do not exclude other moieties, additives, components, integers or steps.

### EXAMPLES

The invention will now be further illustrated by means of the following non-limiting examples.

Abbreviations used in the examples below include:

AR=Liquid Aromatic Phase

AQ=Liquid Aqueous Phase

OR=Liquid Organic Phase

FF=Furfural

LA=Levulinic Acid

AA=Acetic Acid

FA=Formic Acid

HS=Hydrolysate feed solution

T=Temperature

### Example 1

### Extraction of Furfural Using Alkylbenzene as Liquid Aromatic Phase

### (i) Hydrolysate Feed Solution Preparation

A 1000 ml artificial lignocellulosic hydrolysate feed solution (also referred to as “hydrolysate” or HS) was prepared according to Table 1.

TABLE 1
Hydrolysate feed solution for example 1

| Component | Weight in (g) | Grade (%) | wt % |
|---|---|---|---|
| Water | 816.21 | 100 | 81.2 |
| Furfural (FF) | 26.30 | 100 | 2.6 |
| Levulinic acid (LA) | 96.01 | 98 | 9.4 |
| Formic acid (FA) | 36.00 | 98 | 3.5 |
| Acetic acid (AA) | 33.64 | 100 | 3.3 |
| Total | 1008.16 | | 100.0 |

### (ii) Extraction Using Butylbenzene, Pentylbenzene or Hexylbenzene as the Liquid Aromatic Phase:

The hydrolysate (as liquid aqueous phase (AQ)) and an aromatic hydrocarbon compound (forming the liquid aromatic phase (AR)) as indicated in tables 2a, 2b and 2c were mixed together in the following weight portions: 50:25, 50:50 and 50:100 g. The extraction took place in a 500 ml bottle on a stirring device at room temperature (about 20° C.) for 30 minutes. An emulsion was obtained. The emulsion was poured in a separation funnel where a liquid aqueous phase and a liquid organic phase separated quickly, with the formed liquid aqueous phase being the more dense. The two phases were collected in labeled bottles and their contents analyzed.

The above experiments were repeated at 60° C., using a boiling flask with a reflux cooler attached. The temperature was maintained by putting the flask in an oil bath with a temperature controller connected to a hotplate heating the oil.

### Analysis

A mass balance was made for every extraction. Also the distribution ratio (Kd) of furfural (FF) was calculated by dividing the furfural content in the liquid organic phase (wt/wt %) by the furfural content in the liquid aqueous phase (wt/wt %). The distribution ratios (Kd) of levulinic acid (LA), acetic acid (AA) and formic acid (FA) were calculated in an analogous manner.

Water content was determined using a Titroline KF from Schott, a volumetric titrator using the Karl Fischer method. Levulinic acid, formic acid and acetic acid content of the liquid aqueous phase prepared at room temperature was analyzed using an HPLC (Agilent 1100 series). Organic Levulinic acid, formic acid and acetic acid content of the liquid organic phase from the experiments conducted at 60° C. was analyzed using an IEC. Furfural and aromatic hydrocarbon compound content were measured using a GC (Trace GC Ultra).

### Results

The results obtained for extractions using butylbenzene, pentylbenzene and hexylbenzene as the liquid aromatic phase (AR) are presented in Tables 2a-2c respectively. The first row of each table shows the composition of the hydrolysate feed solution (HS) used. Subsequent rows show the results obtained for the different liquid phases after extraction in an alternative fashion (firstly the liquid aqueous phase (AQ) and secondly the liquid organic phase (OR)) for a given liquid aromatic phase/hydrolysate (AR/HS) weight ratio and temperature (T).

TABLE 2a
butylbenzene as the liquid aromatic phase (AR)

| | | | GC analysis | | | HPLC analysis | | | | | | KF |
|---|---|---|---|---|---|---|---|---|---|---|---|---|
| | AR/HS | T | AR | FF | Kd | LA | Kd | FA | Kd | AA | Kd | Water |
| Phase | ratio | (° C.) | (wt %) | (wt %) | (FF) | (wt %) | (LA) | (wt %) | (FA) | (wt %) | (AA) | (wt %) |
| HS | NA | NA | NA | 2.61 | NA | 9.33 | NA | 3.50 | NA | 3.34 | NA | 80.96 |
| AQ | 0.5 | 25 | 0.42 | 1.45 | | 9.51 | | 3.70 | | 3.57 | | 87.94 |
| OR | 0.5 | 25 | 94.26 | 2.18 | 1.50 | 0.09 | 0.01 | 0.00 | 0.00 | 0.00 | 0.00 | 0.04 |
| AQ | 1.0 | 25 | 0.01 | 1.08 | | 9.69 | | 3.78 | | 3.43 | | 87.69 |
| OR | 1.0 | 25 | 94.07 | 1.52 | 1.41 | 0.00 | 0.00 | 0.00 | 0.00 | 0.02 | 0.01 | 0.02 |
| AQ | 2.0 | 25 | 0.23 | 0.72 | | 9.08 | | 3.55 | | 3.27 | | 88.38 |
| OR | 2.0 | 25 | 95.24 | 0.98 | 1.36 | 0.29 | 0.03 | 0.04 | 0.01 | 0.09 | 0.03 | 0.03 |
| AQ | 0.5 | 60 | 0.09 | 1.52 | | 11.29 | | 3.90 | | 3.36 | | 83.90 |
| OR | 0.5 | 60 | 93.29 | 2.19 | 1.44 | 0.00 | 0.00 | 0.00 | 0.00 | 0.14 | 0.04 | 0.04 |
| AQ | 1.0 | 60 | 0.20 | 1.13 | | 11.00 | | 3.83 | | 3.29 | | 89.12 |
| OR | 1.0 | 60 | 94.34 | 1.54 | 1.36 | 0.00 | 0.00 | 0.00 | 0.00 | 0.17 | 0.05 | 0.02 |
| AQ | 2.0 | 60 | 0.31 | 0.74 | | 10.97 | | 3.76 | | 3.21 | | 86.68 |
| OR | 2.0 | 60 | 95.09 | 0.99 | 1.34 | 0.00 | 0.00 | 0.00 | 0.00 | 0.11 | 0.03 | 0.03 |

TABLE 2b
pentylbenzene as the liquid aromatic phase (AR)

| | | | GC analysis | | | HPLC analysis | | | | | | KF |
|---|---|---|---|---|---|---|---|---|---|---|---|---|
| | AR/HS | T | AR | FF | Kd | LA | Kd | FA | Kd | AA | Kd | Water |
| Phase | ratio | (° C.) | (wt %) | (wt %) | (FF) | (wt %) | (LA) | (wt %) | (FA) | (wt %) | (AA) | (wt %) |
| HS | NA | NA | NA | 2.61 | NA | 9.33 | NA | 3.50 | NA | 3.34 | NA | 80.96 |
| AQ | 0.5 | 25 | 0.29 | 1.69 | | 9.62 | | 3.72 | | 3.55 | | 91.53 |
| OR | 0.5 | 25 | 92.07 | 2.05 | 1.21 | 0.00 | 0.00 | 0.00 | 0.00 | 0.00 | 0.00 | 0.06 |
| AQ | 1.0 | 25 | 0.06 | 1.23 | | 9.70 | | 3.78 | | 3.68 | | 92.33 |
| OR | 1.0 | 25 | 93.17 | 1.55 | 1.26 | 0.00 | 0.00 | 0.00 | 0.00 | 0.00 | 0.00 | 0.02 |
| AQ | 2.0 | 25 | 0.53 | 0.84 | | 9.60 | | 3.77 | | 3.51 | | 92.93 |
| OR | 2.0 | 25 | 92.72 | 0.99 | 1.18 | 0.04 | 0.00 | 0.00 | 0.00 | 0.00 | 0.00 | 0.01 |
| AQ | 0.5 | 60 | 0.10 | 1.59 | | 10.92 | | 3.79 | | 3.28 | | 83.23 |
| OR | 0.5 | 60 | 92.23 | 2.09 | 1.31 | 0.00 | 0.00 | 0.00 | 0.00 | 0.29 | 0.09 | 0.03 |
| AQ | 1.0 | 60 | 0.16 | 1.18 | | 10.98 | | 3.81 | | 3.29 | | 84.92 |
| OR | 1.0 | 60 | 91.49 | 1.49 | 1.26 | 0.00 | 0.00 | 0.00 | 0.00 | 0.14 | 0.04 | 0.03 |
| AQ | 2.0 | 60 | 0.24 | 0.80 | | 10.85 | | 3.79 | | 3.22 | | 85.93 |
| OR | 2.0 | 60 | 92.97 | 0.98 | 1.23 | 0.00 | 0.00 | 0.00 | 0.00 | 0.12 | 0.04 | 0.02 |

TABLE 2c
hexylbenzene as the liquid aromatic phase (AR)

| | | | GC analysis | | | HPLC analysis | | | | | | KF |
|---|---|---|---|---|---|---|---|---|---|---|---|---|
| | AR/HS | T | AR | FF | Kd | LA | Kd | FA | Kd | AA | Kd | Water |
| Phase | ratio | (° C.) | (wt %) | (wt %) | (FF) | (wt %) | (LA) | (wt %) | (FA) | (wt %) | (AA) | (wt %) |
| HS | NA | NA | NA | 2.61 | NA | 9.33 | NA | 3.50 | NA | 3.34 | NA | 80.96 |
| AQ | 0.5 | 25 | 0.41 | 1.74 | | 9.58 | | 3.75 | | 3.59 | | 91.86 |
| OR | 0.5 | 25 | 77.94 | 1.76 | 1.01 | 0.00 | 0.00 | 0.00 | 0.00 | 0.00 | 0.00 | 0.02 |
| AQ | 1.0 | 25 | 0.18 | 1.31 | | 9.64 | | 3.81 | | 3.58 | | 92.11 |
| OR | 1.0 | 25 | 89.71 | 1.33 | 1.02 | 0.00 | 0.00 | 0.00 | 0.00 | 0.00 | 0.00 | 0.02 |
| AQ | 2.0 | 25 | 0.35 | 0.90 | | 9.55 | | 3.73 | | 3.51 | | 89.19 |
| OR | 2.0 | 25 | 92.68 | 0.89 | 0.99 | 0.10 | 0.01 | 0.00 | 0.00 | 0.00 | 0.00 | 0.04 |
| AQ | 0.5 | 60 | 0.46 | 1.73 | | 11.36 | | 3.95 | | 3.43 | | 84.94 |
| OR | 0.5 | 60 | 90.52 | 1.81 | 1.05 | 0.00 | 0.00 | 0.00 | 0.00 | 0.00 | 0.00 | 0.02 |
| AQ | 1.0 | 60 | 0.30 | 1.32 | | 11.17 | | 3.88 | | 3.32 | | 84.62 |
| OR | 1.0 | 60 | 91.67 | 1.38 | 1.05 | 0.00 | 0.00 | 0.00 | 0.00 | 0.12 | 0.04 | 0.04 |
| AQ | 2.0 | 60 | 0.27 | 0.88 | | 10.96 | | 3.75 | | 3.20 | | 85.01 |
| OR | 2.0 | 60 | 91.18 | 0.90 | 1.02 | 0.00 | 0.00 | 0.00 | 0.00 | 0.13 | 0.04 | 0.03 |

From the results presented it can be seen that all three alkylbenzenes as liquid aromatic phase were found to have high selectivity for furfural, with Kds of between 1.46 (butylbenzene, 25° C.) and 1.01 (hexylbenzene, 25° C.). The results obtained for extractions performed at 25° C. and 60° C. were similar, indicating that all three alkylbenzenes have low temperature sensitivity. The Kd values for the other components present in the hydrolysate (acetic acid, formic acid, levulinic acid) are all approximately equal to zero. The aromatic hydrocarbon compounds in the liquid aromatic phases used are therefore selective for furfural compared to the other components in the hydrolysate.

(iii) Extraction Using Toluene as the Liquid Aromatic Phase:

Extractions using toluene as the liquid aromatic phase at 25° C. were performed as described above at several weight ratios of liquid aromatic phase (AR) to hydrolysate (HS) and the following results were obtained:

TABLE 3a
toluene as the liquid aromatic phase (AR) at 25° C.

| | AR/HS wt ratio = 0.5 | | |
|---|---|---|---|
| Component | AQ (wt %) | OR (wt %) | Kd |
| Furfural, GLC/FID | 0.895 | 2.700 | 3.017 |
| Levulinic acid, HPLC | 9.565 | 0.074 | 0.008 |
| Formic Acid, HPLC | 4.395 | 0.000 | 0.000 |
| Acetic acid, HPLC | 2.760 | 0.000 | 0.000 |

TABLE 3b
toluene as the liquid aromatic phase (AR) at 25° C.

| | AR/HS wt ratio = 1.0 | | |
|---|---|---|---|
| Component | AQ (wt %) | OR (wt %) | Kd |
| Furfural, GLC/FID | 0.495 | 1.800 | 3.636 |
| Levulinic acid, HPLC | 9.525 | 0.144 | 0.015 |
| Formic Acid, HPLC | 4.400 | 0.000 | 0.000 |
| Acetic acid, HPLC | 2.685 | 0.088 | 0.033 |

TABLE 3c
toluene as the liquid aromatic phase (AR) at 25° C.

| | AR/HS wt ratio = 2.0 | | |
|---|---|---|---|
| Component | AQ (wt %) | OR (wt %) | Kd |
| Furfural, GLC/FID | 0.310 | 1.075 | 3.468 |
| Levulinic acid, HPLC | 9.410 | 0.243 | 0.026 |
| Formic Acid, HPLC | 4.410 | 0.000 | 0.000 |
| Acetic acid, HPLC | 2.575 | 0.128 | 0.050 |

From the results, it can be seen that toluene at 25° C. is also selective for furfural. A distribution coefficient (Kd) for furfural of greater than 3 is obtained but the Kd values for the other hydrolysate components are all approximately equal to zero.

Although toluene as liquid aromatic phase selectively extracts furfural from the hydrolysate (as liquid aqueous phase), more energy will be required as toluene has a lower boiling point than furfural which means that in order to recover the furfural, the toluene volume will have to be evaporated. When using toluene as liquid aromatic phase, the furfural will be taken from the bottom of the distillation column rather than the top (as with the longer chain alkyl benzenes) and so the furfural will be present in a higher concentration and at higher temperatures than if a higher boiling point aromatic hydrocarbon compound was used, which conditions tend to favor the production of undesired furfural condensation products.

### Example 2

### Extraction of Furfural Using Naphthalene in Methylnaphthalene

### (i) Hydrolysate Feed Solution Preparation

A 200 ml artificial lignocellulosic hydrolysate feed solution (also referred to as “hydrolysate” or HS) was prepared having the following composition (see table 4):

TABLE 4
Hydrolysate feed solution for example 2

| Component | Weigh in (g) | Grade (%) | | wt % |
|---|---|---|---|---|
| Water | 163.15 | 100 | | 81.9 |
| Furfural (FF) | 5.41 | 100 | (Merck) | 2.7 |
| Levulinic acid (LA) | 19.20 | 98 | (Sigma Aldrich) | 9.4 |
| Formic acid (FA) | 6.78 | 98 | (Merck) | 3.3 |
| Acetic acid (AA) | 5.47 | 100 | (Merck) | 2.7 |
| Total | 200.01 | | | 100.0 |

### (ii) Preparation of the Liquid Aromatic Phase

A solution of about 20 wt % naphthalene in methylnaphthalene was prepared having the following composition (see table 5):

TABLE 5
Liquid aromatic phase

| Component | Weigh in (g) | Grade (wt %) | | wt % |
|---|---|---|---|---|
| Methylnaphthalene | 149.29 | 94 | (Merck) | 77.7 |
| Naphthalene | 40.61 | 99 | (Sigma Aldrich) | 22.3 |
| Total | 189.90 | | | 100.0 |

(iii) Extraction

The hydrolysate (as liquid aqueous phase) and liquid aromatic phase were mixed together in the following weight proportions: 50:25, 50:50 and 50:100. The extraction was performed in a boiling flask with a reflux cooler, placed in an oil bath on a stirring device, for a period of 30 minutes at a constant temperature of 60° C. An emulsion was obtained.

The emulsion was poured in a separation funnel where a liquid aqueous phase and a liquid organic phase separated quickly, with the formed liquid aqueous phase being the more dense. The two phases were collected in labeled bottles and their contents analyzed.

### (iv) Analysis

The distribution ratio (Kd) of furfural, water content, organic acid content, furfural content and aromatic hydrocarbon compound content were determined according to the methods used in Example 1.

### (v) Results

The results obtained using 21.4% naphthalene in methylnaphthalene as a liquid aromatic phase are presented in Table 6 below.

As for Tables 2a-2c above, the first row of each table shows the composition of the hydrolysate feed solution (HS) used. Subsequent rows show the results obtained for the different liquid phases after extraction in an alternative fashion (firstly the liquid aqueous phase (AQ) and secondly the liquid organic phase (OR)) for a given liquid aromatic phase/hydrolysate (AR/HS) weight ratio and temperature (T).

The Kd obtained for naphthalene in methylnaphthalene (Kd 3.0) is similar to that obtained when toluene is used and is approximately double the value obtained for butylbenzene as liquid aromatic phase. The Kd values of the other components present in the hydrolysate feed solution are zero or almost zero, demonstrating that naphthalene in methylnaphthalene is selective for furfural extraction.

TABLE 6
solution of naphthalene in methylnaphthalene as the liquid aromatic phase (AR)

| | | | GC analysis | | | HPLC analysis | | | | | | KF |
|---|---|---|---|---|---|---|---|---|---|---|---|---|
| | AR/HS | T | AR | FF | Kd | LA | Kd | FA | Kd | AA | Kd | Water |
| Phase | ratio | (° C.) | (wt %) | (wt %) | (FF) | (wt %) | (LA) | (wt %) | (FA) | (wt %) | (AA) | (wt %) |
| HS | NA | NA | NA | 2.70 | NA | 9.41 | NA | 3.32 | NA | 2.73 | NA | 81.57 |
| AQ | 0.5 | 60 | 0.87 | 1.07 | NA | 9.44 | NA | 3.56 | NA | 2.94 | NA | 88.71 |
| OR | 0.5 | 60 | 96.88 | 3.27 | 3.05 | 0.59 | 0.06 | 0.00 | 0.00 | 0.00 | 0.00 | 1.19 |
| AQ | 1.0 | 60 | 1.76 | 0.70 | NA | 9.29 | NA | 3.49 | NA | 2.87 | NA | 92.10 |
| OR | 1.0 | 60 | 95.85 | 2.01 | 2.89 | 0.56 | 0.06 | 0.00 | 0.00 | 0.01 | 0.00 | 1.44 |
| AQ | 2.0 | 60 | 1.10 | 0.40 | NA | 9.35 | NA | 3.57 | NA | 2.85 | NA | 92.84 |
| OR | 2.0 | 60 | 98.61 | 1.17 | 2.93 | 0.38 | 0.04 | 0.01 | 0.00 | 0.05 | 0.02 | 0.81 |

### Comparative Example A

### Extraction of Furfural Using 2-Methyl Tetrahydrofuran (2-MTHF) or Cyclohexanone

### (i) Hydrolysate Feed Solution Preparation

Artificial lignocellulosic hydrolysate feed solution (also referred to as “hydrolysate” or HS) was prepared having the compositions as indicated in Tables 7a, 7b, 7c and 7d.

### (ii) Extraction Using Methyl Tetrahydrofuran or Cyclohexanone:

The hydrolysate (as liquid aqueous phase (AQ)) and 2-methyl tetrahydrofuran (2-MTHF) or cyclohexanone were mixed together in a weight ratio of 2-MTHF or cyclohexanone to hydrolysate as indicated in tables 7a, 7b, 7c and 7d. The extraction took place in a 500 ml bottle on a stirring device at room temperature (about 20° C.) for 30 minutes. An emulsion was obtained. The emulsion was poured in a separation funnel where a liquid aqueous phase (AQ) and a liquid organic phase (OR) separated quickly, with the formed liquid aqueous phase being the more dense. The two phases were collected in labeled bottles and their contents analyzed.

For cyclohexanone the experiment was repeated at 60° C., using a boiling flask with a reflux cooler attached. The temperature was maintained by putting the flask in an oil bath with a temperature controller connected to a hotplate heating the oil.

### (iv) Analysis

The distribution ratio (Kd) of furfural, levulinic acid, formic acid and sulphuric acid and water content, levulinic acid content, formic acid content, sulphuric acid content, furfural content were determined according to the methods used in Example 1.

### (v) Results

The results obtained using 2-MTHF and cyclohexanone to extract furfural are presented in Tables 7a, 7b, 7c and 7d below. As can be seen from Tables 7a, 7b, 7c and 7d, extraction with 2-MTHF or cyclohexanone does not selectively extract the furfural but also extracts considerable amounts of levulinic acid and formic acid.

TABLE 7a
2-Methyl tetrahydrofuran (2-MTHF) to extract furfural at a temperature
of 20° C. and a weight ratio of 2-MTHF to HS of 1.

| | HS | AQ | OR | |
|---|---|---|---|---|
| | % wt. | % wt. | % wt. | Kd |
| 2-MTHF | | 11.52 | 78.42 | |
| Water | 78.96 | 80.69 | 9.67 | |
| Levulinic acid | 10.00 | 3.27 | 6.00 | 1.83 |
| Furfural | 5.04 | 0.58 | 4.06 | 7.03 |
| Formic acid | 3.00 | 0.87 | 1.80 | 2.08 |
| Sulphuric acid | 3.01 | 3.08 | 0.05 | 0.01 |
| total | 100.00 | 100.00 | 100.00 | |

TABLE 7b
2-Methyl tetrahydrofuran (2-MTHF) to extract furfural at a temperature
of 20° C. and a weight ratio of 2-MTHF to HS of 0.50.

| | HS | AQ | OR | |
|---|---|---|---|---|
| | % wt. | % wt. | % wt. | Kd |
| 2-MTHF | | 12.13 | 66.92 | |
| Water | 78.86 | 76.22 | 13.12 | |
| Levulinic acid | 9.98 | 5.47 | 10.06 | 1.84 |
| Furfural | 5.09 | 1.19 | 7.20 | 6.07 |
| Formic acid | 3.04 | 1.47 | 2.57 | 1.76 |
| Sulphuric acid | 3.03 | 3.52 | 0.13 | 0.04 |
| total | 100.00 | 100.00 | 100.00 | |

TABLE 7c
Cyclohexanone to extract furfural at a temperature of 20° C.
and a weight ratio of cyclohexanone to HS of 1.34.

| | HS | AQ | OR | |
|---|---|---|---|---|
| | % wt. | % wt. | % wt. | Kd |
| Cyclohexanone | | 7.45 | 80.50 | |
| Water | 81.57 | 83.78 | 9.68 | |
| Levulinic acid | 9.38 | 3.33 | 5.78 | 1.73 |
| Furfural | 2.60 | 0.19 | 2.08 | 10.88 |
| Formic acid | 3.27 | 1.47 | 1.96 | 1.34 |
| Sulphuric acid | 3.19 | n.d. | n.d. | |
| total | 100.00 | 96.22 | 100.00 | |

TABLE 7d
Cyclohexanone to extract furfural at a temperature of 60° C.
and a weight ratio of cyclohexanone to HS of 1.40.

| | HS | AQ | OR | |
|---|---|---|---|---|
| | % wt. | % wt. | % wt. | Kd |
| Cyclohexanone | | 7.45 | 80.24 | |
| Water | 81.97 | 83.78 | 10.05 | |
| Levulinic acid | 9.43 | 3.33 | 5.81 | 1.73 |
| Furfural | 2.59 | 0.19 | 2.01 | 10.88 |
| Formic acid | 3.29 | 1.47 | 1.88 | 1.34 |
| Sulphuric acid | 2.72 | n.d. | n.d. | |
| total | 100.00 | 96.22 | 100.00 | |

As illustrated above, surprisingly aromatic hydrocarbon compounds can be used to extract furfural selectively from a liquid aqueous phase comprising furfural and one or more organic acids (for example formed by acid catalyzed hydrolysis of biomass), whereas other hydrocarbon compounds are not suitable for such use.

1. A process for separating furfural from a liquid aqueous phase comprising furfural and one or more organic acids, which process comprises a step
a) extracting furfural from the liquid aqueous phase into a liquid aromatic phase comprising one or more aromatic hydrocarbon compounds to obtain a liquid organic phase comprising furfural and one or more aromatic hydrocarbon compounds.

2. The process according to claim 1, wherein the process further comprises a step
b) retrieving furfural from the liquid organic phase obtained in step a) to obtain furfural.

3. The process according to claim 2, wherein the process further comprises a step c) converting the furfural obtained in step b) in a furfural derivative and blending the furfural derivative with one or more other fuel components to produce a biofuel.

4. The process according to claim 1, wherein the liquid aqueous phase comprises one or more organic acids chosen from the group of levulinic acid, acetic acid, formic acid and mixtures thereof.

5. The process according to claim 1, wherein the liquid aqueous phase is produced by acid catalyzed hydrolysis of lignocellulosic material.

6. The process according to claim 1, wherein the liquid aqueous phase comprises more than or equal to 0.1 wt % and less than or equal to 30 wt % of furfural based on the total weight of the liquid aqueous phase.

7. The process according to claim 1, wherein the liquid aqueous phase comprises from more than or equal to 0.1 wt % and less than or equal to 30 wt % of one or more organic acids based on the total weight of the liquid aqueous phase.

8. The process according to claim 1, wherein the liquid aromatic phase comprises one or more aromatic hydrocarbon compounds having a higher boiling point than furfural.

9. The process according to claim 1, wherein the liquid aromatic phase comprises one or more C₁₋₆ alkyl substituted benzenes.

10. The process according to claim 9, wherein the liquid aromatic phase comprises butylbenzene, pentylbenzene, hexylbenzene or a mixture thereof.

11. The process according to claim 1, wherein the liquid aromatic phase comprises a C₁₋₆ alkyl substituted naphthalene.

12. The process according to claim 11, wherein the liquid aromatic phase comprises a mixture of naphthalene and 1-methylnaphthalene.

13. The process according to claim 1, wherein the ratio of the liquid aqueous phase to the liquid aromatic phase is in the range of from 2:1 to 1:5.

14. The process according to claim 1 wherein the process further comprises retrieving one or more aromatic hydrocarbon compounds from the liquid organic phase and recycling the retrieved aromatic hydrocarbon compound(s) to step a).

15. The use of an aromatic hydrocarbon compound to extract furfural selectively from a liquid aqueous phase comprising furfural and one or more organic acids formed by acid catalysed hydrolysis of biomass.
